(19) United States
(12) Patent Application Publication
(10) Pub. No.: US 2006/0063216 A1
(43) Pub. Date: Mar. 23, 2006

(54) Method for diagnosing a predisposition of psychosis in a progeny

(76) Inventor: Eileen Louise Rice McFarland, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/205,777

(22) Filed: Aug. 17, 2005

Related U.S. Application Data
(63) Continuation of application No. 09/724,135, filed on Nov. 28, 2000, now abandoned.

(51) Int. Cl. G01N 33/537; G01N 33/53; G01N 33/543

(52) U.S. Cl. 435/7.92

(57) ABSTRACT

This invention relates to methods for diagnosing a predisposition to psychosis in a progeny who possesses Cw antigen, comprising, obtaining a biological sample from the mother of the progeny; determining the presence of anti Cw antibody in the biological sample, where the presence of an anti Cw antibody in the biological sample is indicative of a predisposition of the progeny to psychosis. Kits for use in the diagnosis of psychosis are also described.

### RELATED APPLICATIONS

This application is a continuation of U.S. application Ser. No. 09/724,135, filed Nov. 28, 2000. The entire teachings of the above application(s) are incorporated herein by reference.

### BACKGROUND OF THE INVENTION

Psychosis is a mental and behavioral disorder causing gross distortion or disorganization of a person's mental capacity, affective response, capacity to recognize reality, communicate and relate to others. The disorder affects the individual to the degree of interfering with the person's capacity to cope with the ordinary demands of everyday life. Psychoses are divided into two major classifications according to their origins: 1) those associated with organic brain syndromes; and 2) those less strictly organic and having some functional components (Stedman's Medical Dictionary, 26^{th} edition, Williams & Wilkins, NY, 1982, M. Spraycar, Ed.).

Schizophrenias, belonging to the later classification, are devastating neuropsychiatric disorders which affect approximately 1% of the population and result in serious disruption in the lives of afflicted individuals and their families. Common symptoms include delusions, conceptual disorganization and visual or auditory hallucinations, as well as changes in affective behavior. The cause or causes of schizophrenia remain obscure. A number of scales for the rating of symptoms and methods for ascertaining the diagnosis have been developed, including the DSM classification by the American Psychiatric Association (Diagnostic and Statistical Manual of Mental Disorders (4th editions), PP. 273-316 (1994)), which have attempted to refine the accuracy of clinical diagnosis. However, it is likely that similar symptoms can result from several underlying abnormalities, and diagnosis relying solely on clinical symptoms is difficult and controversial, as well as subjective, time-consuming and costly. Often the current medications cause the same symptoms that the illness itself causes. Clinical diagnosis must take into consideration many variables when assessing the symptoms of an individual. For instance, culture, age, and gender features. This difficulty in assessment can often result in subjective diagnosis.

Hence, a need exists for a method of diagnosing in a progeny a predisposition of psychosis, particularly schizophrenia, one that does not solely rely on the accuracy of clinical diagnosis as discussed above. Early diagnosis can result in early intervention and better prognosis for the individual.

### SUMMARY OF THE INVENTION

The invention pertains to a method for diagnosing a predisposition to psychosis in a progeny who possesses a Cw blood antigen, comprising, obtaining a biological sample from the mother of the progeny; determining the presence of anti Cw antibody in the biological sample, where the presence of an anti Cw antibody in the biological sample is indicative of predisposition of the progeny to psychosis.

The invention further relates to a kit for use in diagnosis of psychosis comprising a sample of anti-Cw antibody and detector that binds to anti-Cw antibody.

The methods of the present invention provide simple detection of the presence of an antibody to a rare blood factor that is indicative of a predisposition to psychosis. Additionally, these methods do not rely on clinical diagnosis.

### DETAILED DESCRIPTION OF THE INVENTION

The Diagnostic and Statistical Manual of Mental Disorders has classified mental disorders into several axes. Axis I encompasses clinical disorders and covers most of the emotional/behavioral disorders. Axis II relates to personality disorders and metal retardation. Axis III, Axis IV and Axis V encompass the general medical condition, psychosocial and environmental problems and global assessment of functioning, respectively.

The causes and pathogenesis of most mental illnesses have not been determined with certainty. To date, signs and symptoms have been the basis of classification. The defects underlying most disorders are thought to involve genetic, molecular and anatomical changes yet these changes remain elusive.

Genetic predisposition to schizophrenia is an important factor and studying the familial pattern of schizophrenia indicates that the genetic transmission is unusual and not the only cause. Studies conducted on monozygotic twins, where one twin has schizophrenia and the other twin does not, have revealed that although the twins share identical genes they possess anatomical differences in the brain. It is believed that the structural changes are the result of a combined actions of the genes and other factors, perhaps a viral infection, a developmental abnormality or a perinatal injury (Principles of Neural Science, 4^{th} Ed., McGraw-Hill, New York, 2000, Kandel E. R. Ed.).

It is hypothesized that the combination of allelic polymorphisms in the context of a specific genetic background is critical for the disease. Recent studies of schizophrenia have implicated two possible loci that correlate with schizophrenia. One locus is on chromosome 22 and the other is on chromosome 6.

The glycoproteins encoded by the Major Histocompatibility Complex (MHC) located on Chromosome 6 have been extensively studied in both the human and murine systems. Many of the histocompatibility proteins have been isolated and characterized. (Immunology, 3d Ed., W. E. Paul, ed., (Ravens Press N.Y. 1993).

MHC molecules are heterodimeric glycoproteins expressed on cells of higher vertebrates and play a role in immune responses. In humans, these molecules are referred to as human leukocyte antigens (HLA). MHC glycoproteins are divided into two groups, class I and class II, which differ structurally and functionally from each other. In general, the major function of MHC molecules is to bind antigenic peptides and display them on the surface of cells.

Class I MHC molecules are expressed on almost all nucleated cells and are recognized by cytotoxic T lymphocytes, which then destroy the antigen-bearing cells. Class II MHC molecules are expressed primarily on cells involved in initiating and sustaining immune responses, such as T lymphocytes, B lymphocytes, macrophages, and the like. Class II MHC molecules are recognized by helper T lymphocytes and induce proliferation of helper T lymphocytes and amplification of the immune response to the particular antigenic peptide that is displayed.

The MHC Class I of humans on chromosome 6 has three loci, HLA-A, HLA-B, and HLA-C, the first two of which have a large number of alleles encoding alloantigens. Alloantigens consist of a 44 kd subunit and a 12 kd β₂-microglobulin subunit common to all antigenic specificities. These loci are further subdivided into alleles.

Human Class II (encoded by alleles at the HLA-DR, DP, and DQ loci) glycoproteins have a domain structure, including antigen binding sites, similar to that of Class I. The Class II molecules comprise two chains, the α and β chains, which extend from the membrane bilayer. As with the Class I molecules, each subunit in Class II molecules consist of globular domains, referred to as α 1, α 2, β 1, and β2. All except the α 1 domain are stabilized by intrachain disulfide bonds typical of molecules in the immunoglobulin superfamily. The N-terminal portions of the α and β chains, the α 1 and β1 domains, contain hypervariable regions which are thought to comprise the majority of the antigen-binding sites.

As noted above, each MHC allele encodes proteins which comprise hypervariable regions and antigen binding sites specific for particular sets of antigenic peptides. If the peptides bound by the MHC molecule are from an autoantigen, allergen or other protein associated with a deleterious immune response, the hypervariable region of the MHC molecule can be used to produce immunogenic polypeptides which will elicit an immune response against the MHC molecule. HLA genes have been implicated in many immunological disorders and elucidating the HLA associations and role in the pathogenesis of disease would be instrumental in determining a predisposition to various diseases and disorders.

HLA is present on all nucleated cells. Blood group incompatibilities result from a lack of compatibility between two groups of blood cells that are antigenically different because of the presence of an allele or factor in one group and its absence in the other. On erythrocytes, there are the ABO antigen system, the Rh blood group and other less common antigens. The Rh blood group was first identified in Rhesus monkeys and was given the name Rh. There are five antigens in this group: C, D, E, c, and e. Of these, D is the most important and has no complement pair. A person who does not have the D antigen is considered Rh negative. Aside from these common antigens, other rare antigens have also been identified.

Cw is a low frequency antigen that is present in about 2% of the general Caucasian population and between 7 and 9% in Finns, Latvians and Lapps. The Cw antigen is usually inherited in combination with the common Rh haplotype, CDe. The Cw antigen recently has been characterized at the molecular level as an A to G nucleotide substitution in exon 1 on the gene that encodes the common C, c, E, and e Rh antigens. This results in an amino acid change from glutamine to arginine at amino acid 41 of the Rh Ce polypeptide (Reiner et al., Am. J. of Perinatology, 16(6): 277-281 (1999)).

A woman may produce antibodies directed to a factor her progeny possesses due to a transfer of antigen-positive fetal blood, these antibodies then may cross the placenta and attack the fetal blood cells because her immune system recognizes the fetal blood cells as “foreign”. The antibodies affix themselves to the antigen on the cell surface and produce cell lysis. Bilirubin, a component of the heme molecules located in red blood cells is then released into the blood system. This lack of antigen compatibility has been attributed to many problems seen in newborns including jaundice, a condition resulting from the build up of bilirubin. There is an expanding body of research demonstrating a link between pregnancy complications and a propensity for mental illness. Mothers who had contracted influenza in the second trimester of pregnancy had a higher than normal rate of schizophrenia in their progeny.

Rhesus (Rh) incompatibility, a cause of hemolytic disease of the fetus and newborn has been postulated as a risk factor for schizophrenia (Hollister et al., Arch. Gen. Psychiatry, 53:19-24 (1996)). Maternal alloantibodies against the Rh D antigen are known to result in brain damage in humans secondary to hemolytic disease of the fetus and newborn (HDN). Transplacentally acquired maternal erythrocyte alloantibodies cause fetal erythrolysis in the liver and spleen of the fetus and newborn. In severe cases, death may occur in utero as a consequence of hydrops fetalis secondary to acute anemia and associated hypoxia. Post partum, HDN results in an accumulation of bilirubin in the neonate's circulation, which may result in kernicterus, a yellow staining of neuronal elements of the brain, including the basal ganglia and hippocampus. Infants who survive kernicterus often suffer from lasting brain damage.

The first reported case of HDN due to anti-Cw antibody was described in 1947, the report described a nontransfused woman who had 3 infants all of whom died in retrospect from kernicterus prior to the use of exchange therapy. Today, Cw HDN is only of mild to moderate severity and is treated by exchange transfusion and/or phototherapy (Bowman et al., Vox Sang, 64:226-230 (1993)).

The present invention describes a method to detect the predisposition of psychosis resulting from the presence of a rare HLA C allele, Cw antigen, in the progeny of a Cw negative mother, where the mother's immune system has produced an antibody against the progeny's Cw antigen.

In the preferred embodiment, the invention pertains to a method for diagnosing a predisposition to psychosis in a progeny who possesses Cw antigen, comprising, obtaining a biological sample from the mother of the progeny; and determining the presence of anti Cw antibody in the mother's biological sample, where the presence of an anti Cw antibody in the maternal biological sample is indicative of predisposition of the progeny to psychosis. The determination of the anti Cw antibody and Cw antigen can be accomplished by standard methods known in the art, including the antiglobulin Coomb's test, both indirect and direct (Coombs R. R. A et al., Br. J. Exp. Pathol., 26: 255 (1945)). Other standard methods used in tissue and blood typing including typing for organ transplantation could also be used (Vengelen-Tyler V., Ed. Technical Manual, 12^{th} Edition, Bethesda, Md.: American Association of Blood Banks; (1996)). Recently, the use of a polymerase chain reaction (PCR)-based assay for fetal Cw antigen genotyping has been developed which obviates the need for further invasive or noninvasive diagnostic procedures for the remainder of the pregnancy or for prenatal diagnosis in subsequent pregnancies (Reiner et al., Am. J. of Perinatology, 16(6): 277-281 (1999)).

In a further embodiment, psychosis is an axis I or axis II disorder. Axis I disorders include but are not limited to: disorders usually first diagnosed in infancy, childhood or adolescence, delirium, dementia, amnestic, cognitive disorders, mental disorders due to a general medical condition, substance-related disorders, schizophrenia, mood disorders, psychotic disorders, anxiety disorders, somatoform disorders, factitious disorders, dissociative disorders, sexual and gender identity disorders, eating disorders, sleep disorders, impulse-control disorders, adjustment disorders and other conditions that may be a focus of clinical attention. Axis II personality disorders include but are not limited to: paranoid personality disorder, schizoid personality disorder, schizotypal personality disorder, antisocial personality disorder, borderline personality disorder, histrionic personality disorder, narcissistic personality disorder, avoidant personality disorder, dependent personality disorder, obsessive-compulsive personality disorder, and mental retardation.

In yet a further embodiment, the mother and progeny have the same blood type.

The invention further relates to a method of screening for predisposition to psychosis, comprising, obtaining a sample from a maternal donor; and determining the presence of an anti-Cw antibody in the sample, wherein the presence of an anti-Cw antibody is indicative of a predisposition to schizophrenia if donor's progeny possess Cw antigen.

The anti-Cw antibody can be obtained by following the method of Thorpe et al., Vox Sang 73: 174-181:1997. A biological sample is sample from a donor which possess blood antigens or blood antibodies including but not limited to blood, serum and saliva.

A further embodiment includes the progeny having a family history of psychosis or the donor is pregnant or the donor is post-partum. The presence of anti-Cw antibody in a donor who is post-partum is indicative of an histoincompatibility between the donor and progeny and thus a predisposition of the progeny to psychosis.

The invention further relates to a kit for use in diagnosis of psychosis comprising a sample of anti-Cw antibody and detector that binds to anti-Cw antibody.

The invention also relates to a method for diagnosing or aiding in the diagnosis of a predisposition to a psychotic disorder, comprising determining the presence of anti-Cw antibody in a sample from an individual with Cw antigen, wherein the presence of anti-Cw antibody indicates a positive diagnosis.

Case Study

| | | | Antibody | |
|---|---|---|---|---|
| | Blood | Blood | Screening | Family |
| | type | factor | Direct Coombs | history |
| Mother | A+ | A antigens | detected anti-Cw | |
| | | | antibodies | |
| | | | post partum | |
| Father | O | DC/Cw | | Brother and sister |
| | | antigens | | have psychotic |
| | | | | disorders |
| Progeny | A+ | DC/cw antigen/ | | |
| (son, 4^{th} | | A antigens | | |
| child) | | | | |

The mother, age 36, had no previous miscarriage, transfusion, amniocentesis or bleeding during or prior to pregnancy. Anti Cw antibodies were detected during an antibody screen conducted post partum. The mother was diagnosed suffering from pneumonia post partum.

The progeny displayed mild jaundice which is an indication of histoincompatability. The progeny's bilirubin level was 10.2 at birth and rose to 17.2 after 24 hours. The jaundice was treated with phototherapy and according to hospital records no evidence of hemolysis was detected. At age 19, the progeny was diagnosed with schizophrenia. At this time, one white haired facial sideburn was observed which may indicate a biological marker of the onset of schizophrenic illness. One week prior to diagnosis, no brain abnormalities were indicated by an MRI indicating the illness was not a result of a structural brain abnormality.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for diagnosing a predisposition to psychosis in a progeny who possesses Cw blood antigen, comprising:
(a) obtaining a biological sample from the mother of the progeny; and
(b) determining the presence of anti Cw antibody in the biological sample,
wherein the presence of an anti Cw antibody in the biological sample is indicative of predisposition of the progeny to psychosis

2. A method as in claim 1 wherein the psychosis is schizophrenia.

3. A method as in claim 1 wherein the psychosis is an axis I disorder.

4. A method as in claim 1 wherein the psychosis is an axis II disorder.

5. A method as in claim 1 wherein the mother and progeny have the same blood type.

6. A method of screening for predisposition to psychosis, comprising:
(a) obtaining a sample from a maternal donor; and
(b) determining the presence of an anti-Cw antibody in the sample, wherein the presence of an anti-Cw antibody is indicative of a predisposition to schizophrenia if donor's progeny possess Cw antigen.

7. A method as in claim 8 wherein the progeny has a family history of psychosis.

8. A method as in claim 8 wherein the donor is pregnant.

9. A method as in claim 8 wherein the donor is post-partum.

10. A kit for use in diagnosis of psychosis comprising a sample of anti-Cw antibody and detector that binds to anti-Cw antibody.

11. A method for diagnosing or aiding in the diagnosis of a predisposition to a psychotic disorder, comprising determining the presence of anti-Cw antibody in a sample from an individual with Cw antigen, wherein the presence of anti-Cw antibody indicates a positive diagnosis.
